(19) United States
(12) Patent Application Publication
(10) Pub. No.: US 2023/0165660 A1
(43) Pub. Date: Jun. 1, 2023

(54) INSTRUMENT IDENTIFICATION METHOD AND INSTRUMENT IDENTIFICATION SYSTEM

(71) Applicants: YU-PIN LIN, Hsinchu County (TW); GUAN-GUAN LEE, Hsinchu County (TW)

(72) Inventors: YU-PIN LIN, Hsinchu County (TW); GUAN-GUAN LEE, Hsinchu County (TW)

(21) Appl. No.: 17/693,402

(22) Filed: Mar. 13, 2022

(30) Foreign Application Priority Data
Nov. 26, 2021 (TW) 110144095

(51) Int. Cl. A61B 90/90

(52) U.S. Cl. CPC A61B 90/90

(57) ABSTRACT

An instrument identification method and an instrument identification system are provided. The instrument identification method includes detecting a number of a plurality of instruments, and determining whether or not the number of the plurality of instruments is equal to a predetermined number of instruments; detecting instrument image information of one of the plurality of instruments; and determining an instrument code, an instrument type, and appearance integrity of the one of the plurality of instruments according to the instrument image information of a part of each of the plurality of instruments.

### CROSS-REFERENCE TO RELATED PATENT APPLICATION

This application claims the benefit of priority to Taiwan Patent Application No. 110144095, filed on Nov. 26, 2021. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to an instrument identification method and an instrument identification system, and more particularly to an instrument identification method and an instrument identification system which are accurate and simple.

### BACKGROUND OF THE DISCLOSURE

The management and usage monitoring of medical instruments are usually handled manually. However, a usage rate of certain instruments is often much lower than that of other instruments.

Therefore, how to provide an instrument identification system and an instrument identification method that can quickly identify and manage the instruments to overcome the above issues has become one of the important issues to be addressed in the related field.

### SUMMARY OF THE DISCLOSURE

In response to the above-referenced technical inadequacy, the present disclosure provides an instrument identification method and an instrument identification system.

In one aspect, the present disclosure provides an instrument identification method for detecting a plurality of instruments in an instrument box. The instrument identification method includes detecting a number of the plurality of instruments, and determining whether or not the number of the plurality of instruments is equal to a predetermined number of instruments; detecting instrument image information of one of the plurality of instruments; and determining an instrument code, an instrument type, and appearance integrity of one of the plurality of instruments according to a plurality of instrument image information of a part of the one of the plurality of instruments of the instrument image information.

In another aspect, the present disclosure provides an instrument identification system, which includes a controller, a storage device, and an image capture device. The storage device is connected to the controller. The image capture device is connected to the controller and is configured to capture a plurality of image information. The image capture device is configured to capture at least one instrument box image information of an instrument box. The controller is configured to determine a number of a plurality of instruments in the instrument box according to the instrument box image information, and to determine whether or not the number of the plurality of instruments is equal to a predetermined number of instruments. The image capture device is configured to capture instrument image information of one of the plurality of instruments in the instrument box.

Therefore, one of the beneficial effects of the present disclosure is that, in the instrument identification method and the instrument identification system provided by the present disclosure, different instrument types of the instruments, using process of the instruments, and the appearance integrity of the instruments can be effectively identified, so that manage efficiency of the instruments can be improved and manage costs can be reduced.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic view of an instrument identification system according to a first embodiment of the present disclosure;

FIG. 2 is a schematic view illustrating an identification process performed by a controller using a color identification module or an instrument barcode according to the first embodiment of the present disclosure;

FIG. 3 is a schematic view showing that the controller uses instrument image information to identify a plurality of appearance information of instruments according to the first embodiment of the present disclosure; and

FIG. 4 is a flowchart of an instrument identification method according to a second embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.

### First Embodiment

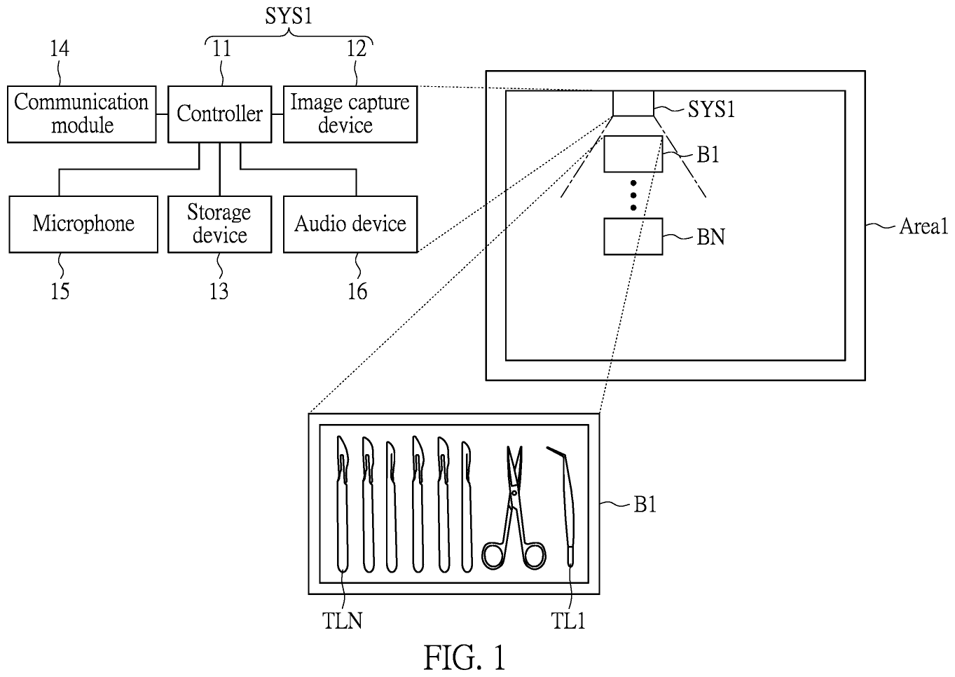

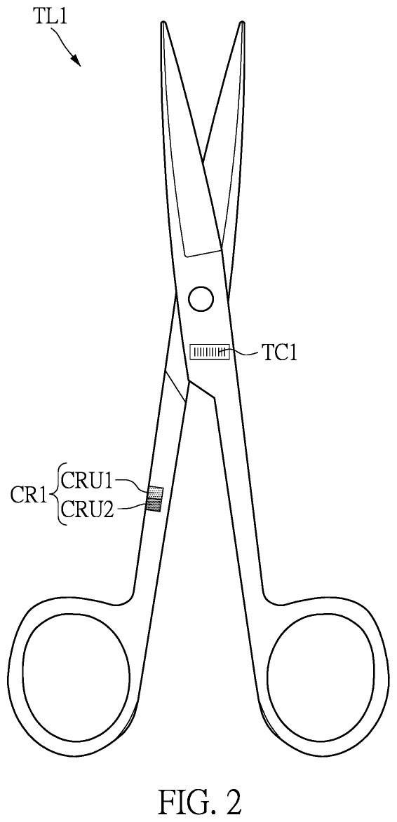

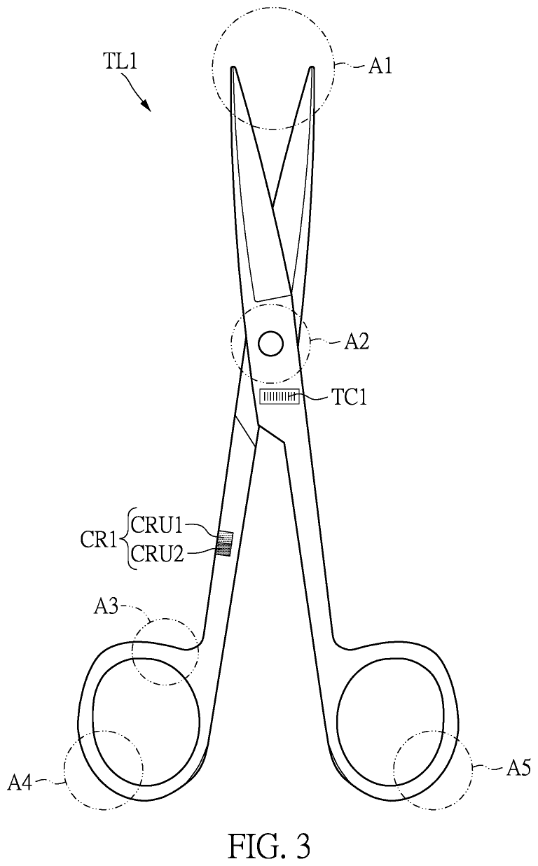

Referring to FIG. 1 to FIG. 3, FIG. 1 is a schematic view of an instrument identification system according to a first embodiment of the present disclosure, FIG. 2 is a schematic view illustrating an identification process performed by a controller using a color identification module or an instrument barcode according to the first embodiment of the present disclosure, and FIG. 3 is a schematic view showing that the controller uses instrument image information to identify a plurality of appearance information of instruments according to the first embodiment of the present disclosure.

The first embodiment provides an instrument identification system SYS1, which includes a controller 11, an image capture device 12, a storage device 13, and a communication module 14.

The controller 11 is correspondingly and electrically connected to the storage device 13, the image capture device 12, and the communication module 14.

The image capture device 12 is configured to capture a plurality of image information.

The image capture device 12 can be used for capturing at least one instrument box image information of an instrument box B1.

The controller 11 can determine a number of instruments and an instrument type of instruments in the instrument box B1 according to the instrument box image information.

That is, the image capture device 12 can also capture the instrument image information of one of a plurality of instruments TL1 to TLN. As shown in FIG. 1, the image capture device 12 can sequentially or selectively capture the instrument image information of one of the plurality of instruments TL1 to TLN in the instrument box B1 to record the number of instruments and the instrument type of instruments in the instrument box B1.

The controller 11 determines a number of the plurality of instruments TL1 to TLN in the instrument box B1 according to the instrument box image information, and determines whether or not the number of the plurality of instruments TL1 to TLN matches a predetermined number of instruments. The controller 11 can also record and store the number of the plurality of instruments TL1 to TLN and the instrument type of instruments in the instrument box B1 in the storage device 13.

The controller 11 detects the instrument type of instruments and an instrument code of each of the plurality of instruments TL1 to TLN, and detects appearance integrity of each of the plurality of instruments TL1 to TLN.

The controller 11 detects the instrument type of instruments and the instrument code of each of the plurality of instruments TL1 to TLN by using the instrument image information of a part of each of the plurality of instruments TL1 to TLN or using a near-field communication device.

A way to use the instrument image information of a part of each of the plurality of instruments TL1 to TLN includes using a barcode and a color identification module that are disposed on each of the plurality of instruments TL1 to TLN. The near-field communication device can be disposed on an outside or an inside of the instrument. That is, in the present embodiment, the controller 11 of the instrument identification system SYS1 can also determine the instrument type of instruments and the instrument code of each of the plurality of instruments TL1 to TLN through the near-field communication devices respectively disposed on the plurality of instruments TL1 to TLN.

In the present embodiment, the image capture device 12 can capture the instrument image information of one of the plurality of instruments TL1 to TLN (e.g., the instrument TL1).

As shown in FIG. 2, the instrument TL1 includes a color identification module CR1.

According to the instrument image information of the instrument TL1, the controller 11 can detect the color identification module CR1 on the instrument TL1 so as to perform an identification process. The controller 11 can determine the instrument code and the instrument type of the instrument TL1 according to the color identification module CR1.

As shown in FIG. 2, the color identification module CR1 has a plurality of color units CRU1, CRU2. In the present embodiment, the plurality of color units CRU1, CRU2 are aligned with each other. In another embodiment, the plurality of color units CRU1, CRU2 can be arranged in a one-dimensional array or a two-dimensional array by a metal coloring process. A number of the color units can be adjusted according to practical requirements, such as four and five.

That is, the controller 11 detects the color identification module CR1 of the instrument TL1 in the instrument image information of the instrument TL1, so as to determine the instrument type of the instrument TL1 and the instrument code corresponding to the instrument TL1. The controller 11 determines the instrument code and the instrument type of the instrument TL1 according to the color identification module CR1.

In the present embodiment, each of the plurality of instruments TL1 to TLN is coded and has a different color identification module disposed thereon, so that identification of the plurality of instruments TL1 to TLN can be properly performed.

In addition, the instrument image information of each of the plurality of instruments TL1 to TLN is captured in advance, and each of the plurality of instruments TL1 to TLN is confirmed to be in good condition.

The color identification module CR1 includes at least two color units CRU1, CRU2. The at least two color units CRU1, CRU2 are correspondingly disposed on a side of the instrument TL1.

As shown in FIG. 2, the at least two color units CRU1, CRU2 are correspondingly and fixedly disposed on at least one surface of the instrument TL1.

In addition, the controller 11 compares the instrument image information of the instrument TL1 with a plurality of predetermined instrument image information stored in the storage device 13 to determine whether or not the instrument image information matches one of the plurality of predetermined instrument image information.

Then, the controller 11 compares a plurality of appearance features A1 to A5 of the instrument image information with a plurality of predetermined appearance features of a corresponding one of the plurality of predetermined instrument image information to determine whether or not the appearance features of the instrument are intact.

The instrument TL1 may be damaged in a using process, which results in defects in appearance of the instrument TL1. Therefore, the instrument image information of the instrument TL1 is compared with each of the plurality of predetermined appearance features to confirm whether or not the instrument TL1 is intact.

Further, the image capture device 12 also detects an instrument barcode TC1 on one surface of the instrument TL1. The instrument barcode TC1 is a one-dimensional barcode or a two-dimensional barcode.

The instrument barcode TC1 is disposed on the one surface of the instrument TL1 by laser engraving.

The instrument barcode TC1 can be disposed on multiple surfaces of the instrument TL1, but the present disclosure is not limited thereto.

In addition, the instrument identification system SYS1 can record the plurality of instruments TL1 to TLN in multiple ones of the instrument boxes B1 to BN at a same location (e.g., a same operating room) but at different points in time (e.g., before and after a surgery), so that the plurality of instruments TL1 to TLN delivered to the same location (e.g., the same operating room) can be accurately compared.

That is, the instrument identification system SYS1 can be disposed in a first area Areal so as to scan the plurality of instruments TL1 to TLN, and to record the plurality of instruments TL1 to TLN entering and leaving the first area Areal.

The plurality of instruments TL1 to TLN can be respectively arranged in the multiple ones of the instrument boxes B1 to BN.

In this way, the plurality of instruments TL1 to TLN entering and leaving the first area Areal can be managed.

In addition, in the present embodiment, the instrument identification system SYS1 further includes a microphone 15. The microphone is connected to the controller 11. The controller 11 and the microphone 15 provide confirmation information of each of the plurality of instruments TL1 to TLN according to sound information and the instrument type as well as the instrument code of each of the plurality of instruments TL1 to TLN. In the present embodiment, the sound information is audio information generated by a user at least according to the instrument type of the plurality of instruments TL1 to TLN in the multiple ones of the instrument boxes B1 to BN. The microphone 15 receives the sound information and transmits the sound information to the controller 11. The controller 11 compares the instrument type of the instrument in the sound information with the instrument type, the instrument code, and appearance integrity of each of the plurality of instruments TL1 to TLN, so as to provide the confirmation information. That is, the user can interact with the instrument identification system SYS by voice or text through the sound information. The microphone 15 receives the sound information of the user, and determines whether or not the instrument type of the instrument in the sound information matches one of the plurality of instruments TL1 to TLN according to the sound information of the user. The instrument identification system SYS1 of the present embodiment can provide an audio device 16 for playing the confirmation information to interact with the user.

In addition, the instrument identification system SYS1 can learn to determine the instrument type and the instrument code of each of the plurality of instruments TL1 to TLN according to the sound information of the user. The confirmation information can be text confirmation information, image confirmation information, or sound confirmation information.

In the present embodiment, the instrument image information of each of the plurality of instruments TL1 to TLN is stored in the storage device 13.

The controller 11 is a central processing unit (CPU), an application specific integrated circuit (ASIC), a graphic processing unit (GPU), or a microcontroller unit (MCU).

The storage device 13 is a flash memory, a read-only memory, a programmable read-only memory, an electrically rewritable read-only memory, an erasable and programmable read-only memory, or an electrically-erasable programmable read-only memory.

The communication module 14 includes a wired communication unit (not shown in the figures) and a wireless communication unit (not shown in the figures). The wired communication unit (not shown in figures) can also be set up independently to communicate with a server (not shown in the figures), so as to receive a control signal from the server (not shown in the figures) or data from a database of the server (not shown in the figures). When the communication module 14 is the wireless communication unit, the communication module 14 can be a Wi-Fi® communication unit, a BLUETOOTH communication unit, a Zigbee communication unit, a LoRa communication unit, a Sigfox communication unit, or an NB-IoT communication unit.

### Second Embodiment

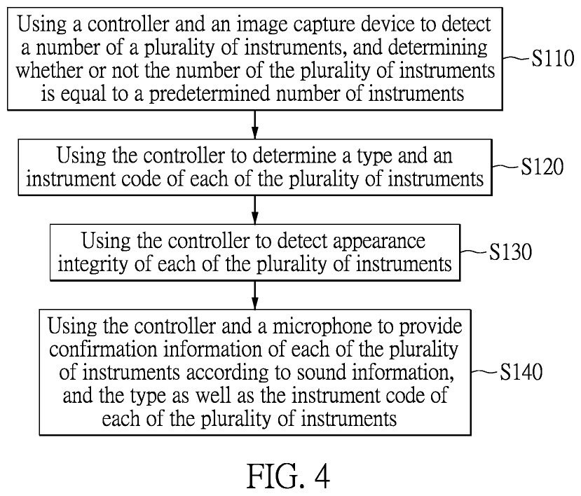

Referring to FIG. 4, FIG. 4 is a flowchart of an instrument identification method according to a second embodiment of the present disclosure.

The present embodiment is applicable to the instrument identification system SYS1 of the first embodiment, and the structure and functions of the instrument identification system SYS1 will not be reiterated herein. The present embodiment provides an instrument identification method for detecting a plurality of instruments in an instrument box. The instrument identification method includes the following steps.

Step S110: using the controller and the image capture device to detect a number of the plurality of instruments, and determining whether or not the number of the plurality of instruments is equal to a predetermined number of instruments;

Step S120: using the controller to determine an instrument type and an instrument code of each of the plurality of instruments;

Step S130: using the controller to detect appearance integrity of each of the plurality of instruments; and

Step S140: using the controller and the microphone to provide confirmation information of each of the plurality of instruments according to sound information, and the instrument type as well as the instrument code of each of the plurality of instruments.

### BENEFICIAL EFFECTS OF THE EMBODIMENTS

In conclusion, one of the beneficial effects of the present disclosure is that, in the instrument identification method and the instrument identification system provided by the present disclosure, different instrument types of the instruments, using process of the instruments, and the appearance integrity of the instruments can be effectively identified, so that manage efficiency of the instruments can be improved and manage costs can be reduced.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. An instrument identification method for an instrument identification system, which includes a controller, an image capture device, and a microphone, and is utilized for detecting a plurality of instruments in an instrument box, the instrument identification method comprising:
using the controller and the image capture device to detect a number of the plurality of instruments, and determining whether or not the number of the plurality of instruments is equal to a predetermined number of instruments;
using the controller to determine an instrument type and an instrument code of each of the plurality of instruments;
using the controller to detect appearance integrity of each of the plurality of instruments; and
using the controller and the microphone to provide confirmation information of each of the plurality of instruments according to sound information, and the instrument type as well as the instrument code of each of the plurality of instruments.

2. The instrument identification method according to claim 1, wherein in the step of using the controller and the image capture device to detect the number of the plurality of instruments, the image capture device is configured to capture at least one instrument image information of the plurality of instruments in the instrument box, and the controller is configured to detect the number of the plurality of instruments according to the at least one instrument image information of the plurality of instruments.

3. The instrument identification method according to claim 2, wherein in the step of using the controller to determine the instrument type and the instrument code of each of the plurality of instruments, the controller is configured to determine the instrument code and the instrument type of each of the plurality of instruments according to the instrument image information of a part of each of the plurality of instruments.

4. The instrument identification method according to claim 3, wherein the instrument image information of the part of each of the plurality of instruments is image information of an instrument barcode.

5. The instrument identification method according to claim 4, wherein the instrument barcode is a one-dimensional barcode or a two-dimensional barcode.

6. The instrument identification method according to claim 3, wherein the instrument image information of the part of each of the plurality of instruments is a color identification module, and the color identification module is configured to determine the instrument code and the instrument type of each of the plurality of the instruments.

7. The instrument identification method according to claim 6, wherein the color identification module includes at least two color units, and the at least two color units are correspondingly disposed on a side of the instrument.

8. The instrument identification method according to claim 7, wherein the at least two color units are correspondingly and movably disposed on the side of the instrument.

9. The instrument identification method according to claim 8, wherein the at least two color units are correspondingly and fixedly disposed on at least one surface of the instrument.

10. The instrument identification method according to claim 2, wherein the instrument image information is compared with a plurality of predetermined instrument image information to determine whether or not the instrument image information matches with one of the plurality of predetermined instrument image information.

11. The instrument identification method according to claim 1, wherein in the step of using the controller to determine the instrument type and the instrument code of each of the plurality of instruments, each of the plurality of instruments further includes a near-field communication device, and the controller determines the instrument type and the instrument code of each of the plurality of instruments according to the near-field communication device of each of the plurality of instruments.

12. The instrument identification method according to claim 3, wherein in the step of using the controller to detect the appearance integrity of each of the plurality of instruments, when the controller obtains multiple ones of the instrument image information of the part of each of the plurality of instruments from multiple ones of the instrument image information, and the multiple ones of the instrument image information of the part of each of the plurality of instruments are a plurality of appearance features of the instrument, the plurality of appearance features of the instrument image information are compared with a plurality of predetermined appearance features of predetermined instrument image information so as to determine whether or not each of the plurality of the appearance features of the instrument is intact.

13. The instrument identification method according to claim 12, wherein in the step of using the controller and the microphone to provide confirmation information of each of the plurality of instruments according to sound information, and the instrument type as well as the instrument code of each of the plurality of instruments, the sound information is audio information generated by a user at least according to the instrument type of the plurality of instruments in the instrument box, the microphone is configured to receive the sound information and transmit the sound information to the controller, and the controller is configured to compare the instrument type of the instrument in the sound information with the instrument type, the instrument code, and the appearance integrity of each of the plurality of instruments obtained in the steps of the instrument identification method, and to provide the confirmation information.

14. The instrument identification method according to claim 14, wherein the confirmation information is text confirmation information, image confirmation information, or sound confirmation information.

15. An instrument identification system, comprising:
a controller;
a microphone connected to the controller;
a storage device connected to the controller; and
an image capture device connected to the controller and being configured to capture a plurality of image information;
wherein the image capture device is configured to capture at least one instrument box image information of an instrument box;
wherein the controller is configured to determine a number of a plurality of instruments in the instrument box according to the instrument box image information, and to determine whether or not the number of the plurality of instruments is equal to a predetermined number of instruments;
wherein the controller is configured to detect an instrument type and an instrument code of each of the plurality of instruments, and to detect appearance integrity of each of the plurality of instruments;
wherein the controller and the microphone are used to provide confirmation information of each of the plurality of instruments according to sound information, and the instrument type as well as the instrument code of each of the plurality of instruments.

16. The instrument identification system according to claim 15, wherein the image capture device is configured to capture at least one instrument image information of the plurality of instruments in the instrument box, and the controller is configured to detect the number of the plurality of instruments according to the at least one instrument image information of the plurality of instruments.

17. The instrument identification system according to claim 16, wherein the controller is configured to determine the instrument code and the instrument type of each of the plurality of instruments according to the instrument image information of a part of each of the plurality of instruments.

18. The instrument identification system according to claim 17, wherein the instrument image information of the part of each of the plurality of instruments is image information of an instrument barcode.

19. The instrument identification system according to claim 18, wherein the instrument barcode is a one-dimensional barcode or a two-dimensional barcode.

20. The instrument identification system according to claim 18, wherein the instrument image information of the part of each of the plurality of instruments is a color identification module, and the color identification module is configured to determine the instrument code and the instrument type of each of the plurality of the instruments.

21. The instrument identification system according to claim 20, wherein the color identification module includes at least two color units, and the at least two color units are correspondingly disposed on a side of the instrument.

22. The instrument identification system according to claim 21, wherein the at least two color units are correspondingly and movably disposed on the side of the instrument.

23. The instrument identification system according to claim 22, wherein the at least two color units are correspondingly and fixedly disposed on at least one surface of the instrument.

24. The instrument identification system according to claim 17, wherein the instrument image information is compared with a plurality of predetermined instrument image information to determine whether or not the instrument image information matches with one of the plurality of predetermined instrument image information.

25. The instrument identification system according to claim 15, wherein each of the plurality of instruments further includes a near-field communication device, and the controller determines the instrument type and the instrument code of each of the plurality of instruments according to the near-field communication device of each of the plurality of instruments.

26. The instrument identification system according to claim 25, wherein, when the controller obtains multiple ones of the instrument image information of the part of each of the plurality of instruments from multiple ones of the instrument image information, and the multiple ones of the instrument image information of the part of each of the plurality of instruments are a plurality of appearance features of the instrument, the plurality of appearance features of the instrument image information are compared with a plurality of predetermined appearance features of predetermined instrument image information so as to determine whether or not each of the plurality of the appearance features of the instrument is intact.

27. The instrument identification system according to claim 26, wherein, when the controller and the microphone are configured to provide confirmation information of each of the plurality of instruments according to sound information, and the instrument type as well as the instrument code of each of the plurality of instruments, the sound information is audio information generated by a user at least according to the instrument type of the plurality of instruments in the instrument box, the microphone is configured to receive the sound information and transmit the sound information to the controller, the controller is configured to compare the instrument type of the instrument in the sound information with the instrument type, the instrument code, and the appearance integrity of each of the plurality of instruments obtained in the steps of the instrument identification method, and to provide the confirmation information.

28. The instrument identification system according to claim 27, wherein the confirmation information is text confirmation information, image confirmation information, or sound confirmation information.
